(19) United States
(12) Patent Application Publication
(10) Pub. No.: US 2023/0250049 A1
(43) Pub. Date: Aug. 10, 2023

(54) PROCESS FOR THE PREPARATION OF ORTHO-HALOGENATED PHENYLALANINE COMPOUNDS

(71) Applicants: CNRS, PARIS Cédex 16 (FR); L'INSTITUT NATIONAL DES SCIENCES APPLIQUEES DE ROUEN, Saint-Etienne-Du-Rouvray Cedex (FR); UNIVERSITE DE ROUEN, MONT ST AIGNAN CEDEX (FR); LES LABORATOIRES SERVIER, SURESNES (FR)

(72) Inventors: Jean-François BRIERE, AMFREVILLE-LA-MIVOIE (FR); Guillaume JOURNOT, Le Bouveret Valais (CH); Sébastien COUFOURIER, Bois Guillaume (FR); Alexia VILLE, Mont Saint Aignan (FR); Julien ANNIBALETTO, Viroflay (FR); Cédric SCHNEIDER, Bose Guérard Saint Adrien (FR); Christophe HOARAU, Bois Guillaume (FR)

(21) Appl. No.: 18/002,917

(22) PCT Filed: Jun. 22, 2021
(86) PCT No.: PCT/EP2021/066938

§ 371 Date: Dec. 22, 2022

(30) Foreign Application Priority Data
Jun. 23, 2020 (EP) 20315311.9

(51) Int. Cl. C07C 227/14; B01J 23/44; C07D 209/26

(52) U.S. Cl. CPC C07C 227/14; B01J 23/44; C07D 209/26

(57) ABSTRACT

Process of ortho-halogenation of phenylalanine compounds by C—H activation.

The present invention relates to a process for the preparation of ortho-halogenated phenylalanine compounds by C—H activation.

Ortho-halogenated (L)-phenylalanine derivatives are particularly useful in the synthesis of (S) indoline-2-carboxylic acid, a key intermediate in the preparation of perindopril and indolapril.

Perindopril, indolapril, and their pharmaceutically acceptable salts, have valuable pharmacological properties.

Their principal property is that of inhibiting angiotensin I converting enzyme, which makes it possible to prevent, on the one hand, conversion of the decapeptide angiotensin I to the octapeptide angiotensin II (a vasoconstrictor) and, on the other hand, degradation of bradykinin (a vasodilator) to an inactive peptide.

Those two actions contribute to the beneficial effects of perindopril and indolapril in cardiovascular diseases, more especially in arterial hypertension, heart failure and stable coronary disease.

Processes for the preparation of (S)-indoline-2-carboxylic acid, a key intermediate in the preparation of perindopril and indolapril, have been described, for example in EP 0 308 339, CN1709871 and EP 1 833 789.

EP 1 833 789 discloses the preparation of (S) indoline-2-carboxylic acid by cyclisation of an ortho-halogenated L-phenylalanine derivative. The ortho-halogenated L-phenylalanine compound is prepared by reaction of an ortho-halogenated cinnamic acid with an amino group donor in the presence of a stereoselective Phenylalanine Ammonia Lyase enzyme (PAL). The ortho-halogenated cinnamic acid is not commercially available and has to be prepared, for example, from an ortho-halogenated benzaldehyde.

It is particularly advantageous to prepare the ortho-halogenated derivative of phenylalanine from phenylalanine because of its availability and low cost.

The patent application CN1709871, as well as the publications Chem. Pharm. Bull. 2006 54, 1715 and Angew. Chem. Int. Ed. 2007, 46, 1281 disclose halogenation reactions of L-phenylalanine or derivatives thereof, but the obtained compound is either dihalogenated, or monohalogenated in the form of a mixture of ortho- and para-halogenated compounds.

More specifically, CN1709871 discloses the chlorination of L-phenylalanine to lead to 2,4-dichloro-L-phenylalanine, ie the ortho, para-dichloro compound. The process of CN1709871 is not a selective ortho-halogenation process.

An example of C—H ortho-halogenation of a phenylalanine derivative has been disclosed (Organometallics 2007, 26, 2768), but the process uses a stoichiometric amount of palladium and needs several synthesis steps.

The problem of the present invention was to find a selective ortho-halogenation process of phenylalanine, in order to obtain the corresponding ortho-halogenated compounds in a good yield and with excellent purity.

More specifically, the present invention relates to a process for the preparation of the compound of formula (A), the compound of formula (B), or a mixture thereof:

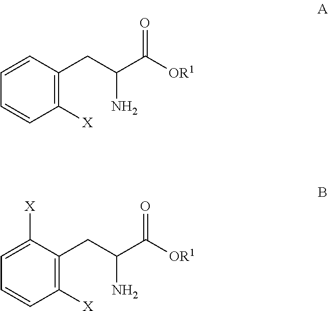

wherein R¹ is a hydrogen atom, a benzyl group or a C₁-C₆ linear or branched alkyl group, and X is a halogen atom selected from Cl, Br and I,
by reaction of the compound of formula (I):

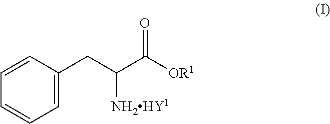

wherein R¹ is as defined before, and HY¹ is an acid,
with a halogenating agent, in the presence of a palladium catalyst and an acid HY², in an organic solvent or a mixture of organic solvents.

The palladium catalyst is preferably used in a substoichiometric amount.

Depending on the conditions, more especially on the nature and amount of halogenating agent and palladium species, on the nature of the solvent and acids, the process may lead to the compound of formula (A), to the compound of formula (B), or to a mixture thereof.

In accordance with an embodiment of the present invention, the process leads to a mixture of the monohalogenated compound of formula (A) and dihalogenated compound of formula (B). The monohalogenated and dihalogenated compounds may be separated, for example by reversed-phase chromatography, or by benzoylation of the mixture, separation by chromatography of the monohalogenated and dihalogenated N-benzoyl compounds, followed by their deprotection.

In accordance with another embodiment of the present invention, the process leads to the monohalogenated compound of formula (A).

In accordance with another embodiment of the present invention, the process leads to the dihalogenated compound of formula (B).

Suitable halogenating agents are for example N-bromosuccinimide, N-chlorosuccinimide, N-iodosuccinimide, N-bromophtalimide, N-chlorophtalimide, 1,3 -dibromo-5,5-dimethylhydantoin and 2-chloro-1,3-bis(methoxycarbonyl)guanidine.

In accordance with a preferred embodiment of the present invention, X is Cl or Br. The halogenating agent is preferably N-bromosuccinimide, N-chlorosuccinimide, N-bromophtalimide or N-chlorophtalimide.

In order to obtain the mono-halogenated compound of formula (A), alone or in mixture with the dihalogenated compound of formula (B), the amount of halogenating agent is preferably from 1 to 2 mole per mole of compound of formula (I), more preferably from 1 to 1.5 mole per mole of compound of formula (I).

In order to obtain the dihalogenated compound of formula (B), the amount of halogenating agent is preferably from 1 to 3 mole per mole of compound of formula (I), more preferably around 2 per mole of compound of formula (I).

In accordance with an embodiment of the present invention, R¹ is methyl or ethyl.

In accordance with another embodiment of the present invention, HY¹ is hydrochloric acid, acetic acid, trifluoroacetic acid or trifluoromethanesulfonic acid.

HY¹ is preferably trifluoroacetic acid.

In accordance with another embodiment of the present invention, the palladium catalyst is a divalent palladium catalyst, preferably palladium(II) acetate. The catalytic amount is preferably from 2.5 to 20 mol %, for example about 10 mol %.

In accordance with another embodiment of the present invention, HY² is trifluoroacetic acid or trifluoromethanesulfonimide.

HY² is preferably trifluoroacetic acid.

The amount of acid HY² is preferably from 2.5 to 20 moles per mole of compound of formula (I), more preferably from 5 to 15 moles per mole of compound of formula (I).

In accordance with another embodiment of the present invention, the reaction is conducted in the presence of a metal additive, preferably a copper(I) or copper(II) catalyst, for example copper(I) chloride, copper(II) chloride, copper(I) bromide, copper(II)bromide, copper(I)iodide, copper(II) iodide, basic copper(II) carbonate, copper(I) nitrate, copper(II) nitrate, copper(II) sulphate, copper(I) sulfide, copper(II) sulfide, copper(I) acetate, copper(II) acetate, copper(I) oxide, copper(II) oxide, copper(I) trifluoroacetate, copper(II) trifluoroacetate, copper(I) benzoate, copper(II) benzoate, copper(II) trifluoromethylsulfonate, more preferably copper(II) acetate.

The amount of metal additive is preferably from 0.2 to 1.2 mole per mole of compound of formula (I), more preferably about 1 mole per mole of compound of formula (I).

In accordance with another embodiment of the present invention, the organic solvent is selected from dichloromethane, dichloroethane, hexafluoroisopropanol, trifluorotoluene, chlorobenzene, trifluoroacetic acid, or a mixture thereof.

In order to obtain the mono-halogenated compound of formula (A), alone or in mixture with the dihalogenated compound of formula (B), the organic solvent is preferably a mixture of dichloromethane and hexafluoroisopropanol.

The ratio dichloromethane/hexafluoroisopropanol is preferably from 9/1 à 1/3 V/V, more preferably about 1/1 V/V.

In order to obtain the dihalogenated compound of formula (B), the solvent is preferably trifluoroacetic acid.

In accordance with another embodiment of the present invention, the temperature of the reaction is from 30° C. to 80° C., preferably from 40 to 60° C.

The compound of formula (I) may be prepared in situ by reaction of the free amine of formula (II):

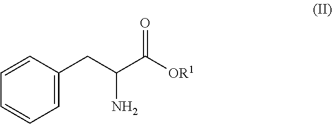

with the acid HY¹.

The stereochemistry of the starting material is retained during the process of the present invention.

In accordance with an embodiment of the present invention, the starting material of formula (I) is of (S) configuration, ie the compound of formula (I) is a derivative of (L)-phenylalanine, leading to (A) and (B) in (S) configuration.

In accordance with another embodiment of the present invention, the starting material of formula (I) is of (R) configuration, leading to (A) and (B) in (R) configuration.

In accordance with another embodiment of the present invention, the starting material of formula (I) is racemic, leading to racemic (A) and (B).

The ortho mono-halogenated and dihalogenated phenylalanine derivatives of formula (A) and (B) may advantageously be used as reactants in the preparation of indoline-2-carboxylic acid derivatives of formula (IIIA) and (IIIB):

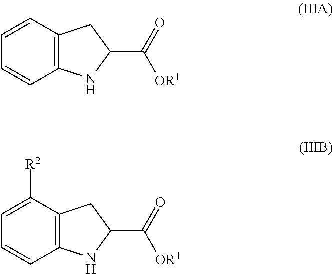

wherein R¹ is as defined before, and R² is Cl, Br or I,

by an intramolecular aryl amination reaction, using for example the conditions disclosed in EP 1 833 789.

The compound of formula (IIIB) may be reduced into the corresponding compound of formula (IIIA), using for example the conditions disclosed in CN1709871.

The following examples illustrate the invention.

Abbreviations:

Bn Benzyl

DCE Dichloroethane

DCM Dichloromethane

Et Ethyl

HFIP Hexafluoroisopropanol

iPr Isopropyl

Me Methyl

NBS N-Bromosuccinimide

NCS N-Chlorosuccinimide

PE Petroleum ether

Phe Phenylalanine

TFA Trifluoroacetic acid

General Information

Reactions were performed using oven dried glassware (without inert conditions). Unless otherwise noted, all reagent-grade chemicals and solvents were obtained from commercial suppliers and were used as received. N-bromo and N-chloro-succinimide were recrystallized in water from commercial batches. Reactions were monitored by thin-layer chromatography with silica gel 60 F254 pre-coated aluminium plates (0.25 mm). Visualization was performed under UV light. Chromatographic purification of compounds was achieved with 60 silica gel (40-63 μm) according to Still, W. C.; Kahn, M.; Mitra, A. J. Org. Chem. 1978, 43, 2923. Melting points were measured on a WME Köfler hot-stage (Stuart SMP3) and are uncorrected. Infrared spectra (IR) were recorded on a PerkinElmer Spectrum 100 Series FT-IR spectrometer. Liquids and solids were applied on the Single Reflection Attenuated Total Reflectance (ATR) Accessories. Data are reported in cm⁻¹. ¹H Spectra (300 MHz) and ¹³C NMR spectra (75 MHz) were recorded on a Bruker Avance 300. Data appear in the following order: chemical shifts in ppm which were referenced to the internal solvent signal, multiplicity (s, singlet; d, doublet; t, triplet; q, quadruplet; m, multiplet, AB, AB system; br, broad), coupling constant J in Hertz and number of protons. Accurate Mass measurements (HRMS) were performed by the Mass Spectrometry Laboratory of the University of Rouen and using a Waters LCT Premier XE mass spectrometer. Accurate Mass measurements (HRMS) were recorded with a Waters LCP 1er XR spectrometer.

### Example 1: Methyl (S)-2-amino-3-(2-bromophenyl)propanoate 1A—Mixture with Methyl (S)-2-amino-3-(2,6-dibromophenyl)propanoate 1B

A mixture of DCM/HFIP (3 mL, 1:1) solvents was added to palladium acetate (7 mg, 0.03 mmol, 10 mol %) and copper acetate (54 mg, 0.3 mmol, 1 equiv.) into an oven dried tube. NBS (80 mg, 0.45 mmol, 1.5 equiv.), L-Phe-OMe.HTFA (88 mg, 0.3 mmol, 1 equiv.) and trifluoroacetic acid (230 μL, 3 mmol, 10 equiv.) were added to the reaction mixture. The tube was sealed and the reaction was stirred at 50° C. (oil bath temperature) for 16 hours. After cooling to room temperature, the reaction mixture was diluted by DCM (20 mL), quenched with a saturated aqueous solution of Na₂CO₃ (5 mL) and the two layers were separated. The aqueous phase was extracted once with dichloromethane (10 mL). The combined organic layers were washed with a saturated aqueous solution of Na₂CO₃ (5 mL), a solution of brine (5 mL) and dried over Na₂SO₄ before filtration. Bn₂O (14.4 μL, 0.25 equiv.) was added as the internal standard for the determination of the yield by ¹H NMR (67% 1A, 34% 1B) if required. The solution was concentrated under vacuum to afford the crude product.

### Benzoylation: Methyl (S)-2-benzamido-3-(2-bromophenyl)propanoate 1C and Methyl (S)-2-benzamido-3-(2,6-dibromophenyl)propanoate 1D

The crude 1A and 1B mixture was solubilized in DCM (3 mL) and a saturated aqueous solution of K₂CO₃ (3 mL) and benzoic anhydride (136 mg, 0.6 mmol, 2 equiv. based on starting 1A and 1B mixture) was added. The tube was sealed and the reaction was vigorously stirred (1200 rpm) at room temperature for 2 hours. The mixture was diluted with DCM (20 mL), washed with water (5 mL), a saturated aqueous solution of Na₂CO₃ (5 mL) and eventually a solution of brine (5 mL). The organic layer was dried over Na₂SO₄, filtered and evaporated under vacuum. The yields were measured by ¹H-NMR spectroscopy by means of Bn₂O as an internal standard (49% 1C, 39% 1D). The crude reaction mixture was purified by flash column chromatography on silica gel (petroleum ether/ethyl acetate 9:1 to 8:2) to afford 1C as a white solid (50 mg, 46% over two-steps) and 1D as a white solid (50 mg, 38% over two-steps).

Methyl (S)-2-benzamido-3-(2-bromophenyl)propanoate 1C:

R_{f}=0.16 (PE/AcOEt: 9/1).

¹H NMR (300 MHz; CDCl₃) δ_{H} 7.75-7.72 (m, 2H), 7.57-7.38 (m, 4H), 7.28-7.21 (m, 2H), 7.15-7.08 (m, 1H), 6.67 (brd, J=7.7 Hz, 1H), 5.14-5.07 (m, 1H), 3.76 (s, 3H), 3.44 (dd, J=6.1, 14.0 Hz, 1H), 3.34 (dd, J=7.6, 14.0 Hz, 1H). ¹³C NMR (75 MHz; CDCl₃) δ_{C} 172.2 (C), 167.1 (C), 136.1 (C), 133.8 (C), 133.1 (CH), 131.3 (CH), 131.4 (CH), 129.0 (CH), 128.7 (CH), 127.8 (CH), 127.2 (CH), 125.1 (C), 53.2 (CH or CH₃), 52.7 (CH or CH₃), 38.0 (CH₂).

HRMS (ESI⁺): calculated for C₁₇H₁₇BrNO₃ [(M+H)⁺]: 362.0386; found: 362.0394.

### Example 2: Methyl (S)-2-amino-3-(2-bromophenyl)propanoate 1A—Mixture with Methyl (S)-2-amino-3-(2,6-dibromophenyl)propanoate 1B

A mixture of DCM/HFIP (10 mL, 1:1) solvents was added to palladium acetate (22 mg, 0.1 mmol, 10 mol %) in an open vessel or a sealed tube. NBS (196 mg, 1.1 mmol, 1.1 equiv.), L-Phe-OMe.HTFA (293 mg, 1 mmol, 1 equiv.) and trifluoroacetic acid (766 μL, 10 mmol, 10 equiv.) were added to the reaction mixture. The reaction was stirred at 50° C. (oil bath temperature) for 16 hours. After cooling to room temperature, the reaction mixture was diluted with DCM (60 mL), quenched with a saturated aqueous solution of Na₂CO₃ (15 mL) and the two layers were separated. The aqueous phase was extracted once with dichloromethane (20 mL). The combined organic layers were washed with a saturated aqueous solution of Na₂CO₃ (15 mL), then a solution of brine (15 mL) and dried over Na₂SO₄ before filtration. Bn₂O (48 μL, 0.25 equiv.) was added as the internal standard for the determination of the yield by ¹H NMR (sealed tube: 57% 1A, 21% 1B, open vessel: 54% 1A, 20% 1B). The solution was concentrated under vacuum to afford the crude product.

### Example 3: Methyl (S)-2-amino-3-(2,6-dibromophenyl)propanoate 1B

TFA (3 mL) solvent was added to palladium acetate (14 mg, 0.06 mmol) into an oven dried tube. NBS (107 mg, 0.6 mmol, 2 equiv.) and L-Phe-OMe.HTFA (88 mg, 0.3 mmol, 1 equiv.) were added to the reaction mixture. The tube was sealed and the reaction was stirred at 50° C. (oil bath temperature) for 16 hours. After cooling to room temperature, the reaction mixture was diluted with DCM (20 mL), quenched with a saturated aqueous solution of Na₂CO₃ (40 mL) and the two layers were separated. The aqueous phase was extracted once with dichloromethane (10 mL). The combined organic layers were washed with a saturated aqueous solution of Na₂CO₃ (5 mL), a solution of brine (5 mL) and dried over Na₂SO₄ before filtration. Bn₂O (14.4 μL, 0.25 equiv.) was added as the internal standard for the determination of the yield by ¹H NMR (72% 1B) if required. The solution was concentrated under vacuum to afford 1B as a crude product.

### Benzoylation: Methyl (S)-2-benzamido-3-(2,6-dibromophenyl)propanoate 1D

The crude 1B compound was solubilized in DCM (3 mL), and a saturated aqueous solution of K₂CO₃ (3 mL) and benzoic anhydride (136 mg, 0.6 mmol, 2 equiv. based on crude 1B) was added to the reaction medium. The tube was sealed and the reaction was vigorously stirred (1200 rpm) at room temperature for 2 hours. The reaction mixture was diluted with DCM (20 mL), washed with water (5 mL), a saturated aqueous solution of Na₂CO₃ (5 mL) and eventually a solution of brine (5 mL). The organic layer was dried over Na₂SO₄, filtered and evaporated under vacuum. The yield was measured by ¹H-NMR spectroscopy by means of Bn₂O as an internal standard (69% 1D). The crude reaction mixture was purified by flash column chromatography on silica gel (petroleum ether/ethyl acetate 9:1 to 8:2) to afford 1D as a white solid (86 mg, 65%).

R_{f}=0.19 (PE/AcOEt: 9/1).

¹H NMR (300 MHz; CDCl₃) δ_{H} 7.78-7.73 (m, 2H), 7.53-7.45 (m, 3H), 7.43-7.37 (m, 2H), 6.95 (t, J=8.0 Hz, 1H), 6.82 (brd, J=8.4 Hz, 1H), 5.42-5.24 (m, 1H), 3.79 (s, 3H), 3.65-3.54 (m, 2H). ¹³C NMR (75 MHz; CDCl₃) δ_{C} 172.2, 167.1, 135.7, 133.7, 132.6, 131.9, 129.9, 128.6, 127.2, 126.2, 52.9, 51.6, 38.9. HRMS (ESI⁺): calculated for C₁₇H₁₆⁷⁹Br₂NO₃ [(M+H)⁺]: 439.9491; found: 439.9497.

### Example 4: Methyl (S)-2-amino-3-(2-chlorophenyl)propanoate 2A—Mixture with Methyl (S)-2-amino-3-(2,6-dichlorophenyl)propanoate 2B

A mixture of DCM/HFIP (3 mL, 1:1) solvents was added to palladium acetate (7 mg, 0.03 mmol, 10 mol %) into an oven dried tube. NCS (60 mg, 0.45 mmol, 1.5 equiv.), L-Phe-OMe. HTFA (88 mg, 0.3 mmol, 1 equiv.) and trifluoroacetic acid (230 μL, 3 mmol, 10 equiv.) were added to the reaction mixture. The tube was sealed and the reaction was stirred at 50° C. (oil bath temperature) for 16 hours. After cooling to room temperature, the reaction mixture was diluted in DCM (20 mL), quenched with a saturated aqueous solution of Na₂CO₃ (5 mL) and the two layers were separated. The aqueous phase was extracted once with dichloromethane (10 mL). The combined organic layers were washed with a saturated aqueous solution of Na₂CO₃ (5 mL), then a solution of brine (5 mL) and dried over Na₂SO₄ before filtration. Bn₂O (14.4 μL, 0.25 equiv.) was added as the internal standard for the determination of the yield by ¹H NMR (78% 2A, 27% 2B) if required. The solution was concentrated under vacuum to afford the crude product.

### Benzoylation: Methyl (S)-2-benzamido-3-(2-chlorophenyl)propanoate 2C and Methyl (S)-2-benzamido-3-(2,6-dichlorophenyl)propanoate 2D

The crude 2A and 2B mixture was solubilized in DCM (3 mL), and a saturated aqueous solution of K₂CO₃ (3 mL) and benzoic anhydride (136 mg, 0.6 mmol, 2 equiv. based on crude 2A and 2B mixture) was added to the reaction medium. The tube was sealed and the reaction was vigorously stirred (1200 rpm) at room temperature for 2 hours. The reaction mixture was diluted with DCM (20 mL), washed with water (5 mL), a saturated aqueous solution of Na₂CO₃ (5 mL) and eventually a solution of brine (5 mL). The organic layer was dried over Na₂SO₄, filtered and evaporated under vacuum. The yields were measured by ¹H-NMR spectroscopy by means of Bn₂O as an internal standard (61% 2C, 26% 2D). The crude reaction mixture was purified by flash column chromatography on silica gel (petroleum ether/ethyl acetate 9:1 to 8:2) to afford 2C as a white solid (55 mg, 58%) and 2D as a white solid (31 mg, 29%).

Methyl (S)-2-benzamido-3-(2-chlorophenyl)propanoate 2C: R_{f}=0.18 (PE/AcOEt: 9/1). ¹H NMR (300 MHz; CDCl₃) δ_{H} 7.76-7.72 (m, 2H), 7.53-7.35 (m, 4H), 7.27-7.17 (m, 3H), 6.66 (brd, J=7.5 Hz, 1H), 5.13-5.06 (m, 1H), 3.76 (s, 3H), 3.43 (dd, J=6.1, 13.9 Hz, 1H), 3.34 (dd, J=7.3, 13.9 Hz, 1H). ¹³C NMR (75 MHz; CDCl₃) δ_{C} 172.2 (C), 167.1 (C), 134.6 (C), 134.3 (C), 133.9 (C), 131.9 (CH), 131.6 (CH), 129.8 (CH), 128.8 (CH), 128.7 (CH), 127.2 (CH), 53.2 (CH or CH₃), 52.7 (CH or CH₃), 35.6 (CH₂).

Methyl (S)-2-benzamido-3-(2,6-dichlorophenyl)propanoate 2D : R_{f}=0.21 (PE/AcOEt: 9/1). ¹H NMR (300 MHz; CDCl₃) δ_{H} 7.70-7.66 (m, 2H), 7.47-7.41 (m, 1H), 7.38-7.32 (m, 2H), 7.25-7.19 (m, 2H), 7.07 (dd, J=8.6, 7.5 Hz, 1H), 6.72 (d, J=8.3 Hz, 1H), 5.24-5.15 (m, 1H), 3.73 (s, 3H), 3.48 (dd, J=6.3, 13.7 Hz, 1H), 3.42 (dd, J=9.4, 13.7 Hz, 1H). ¹³C NMR (75 MHz; CDCl₃) δ_{C} 172.3, 167.2, 136.1, 133.8, 132.9, 131.9, 129.1, 128.7, 128.5, 127.2, 52.9, 51.6, 33.8. HRMS (ESI⁺): calculated for C₁₇H₁₆³⁵Cl₂NO₃ [(M+H)⁺]: 352.0502; found: 352.0503.

### Example 5: Preparation of Compounds 3-9

A mixture of DCM/HFIP (1:1) solvents was added to palladium acetate (10 mol %) in a sealed tube. NXS (1.1 equiv.), L-Phe-OR¹.HTFA (1 equiv.) and trifluoroacetic acid (10 equiv.) were added to the reaction mixture. With NBS or NCS, the reaction was stirred at 50° C. (oil bath temperature) for 16 hours; with NIS, the reaction was stirred at 40° C. After cooling to room temperature, the reaction mixture was diluted with DCM, quenched with a saturated aqueous solution of Na₂CO₃ and the two layers were separated. The aqueous phase was extracted once with dichloromethane. The combined organic layers were washed with a saturated aqueous solution of Na₂CO₃, then a solution of brine and dried over Na₂SO₄ before filtration. An internal standard was used for the determination of the yield by ¹H NMR.

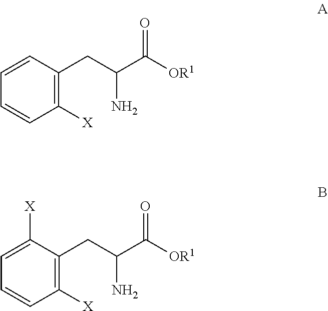

| Compound | R¹ | X | Ratio A:B (RMN ¹H) |
|---|---|---|---|
| 3 | Bn | Br | 56:21 |
| 4 | Bn | Cl | 62:26 |
| 5 | Et | Br | 66:20 |
| 6 | Et | Cl | 78:15 |
| 7 | iPr | Br | 48:23 |
| 8 | iPr | Cl | 43:21 |
| 9 | Me | I | 50:11 |

1. A process for the preparation of the compound of formula (A), the compound of formula (B), or a mixture thereof: 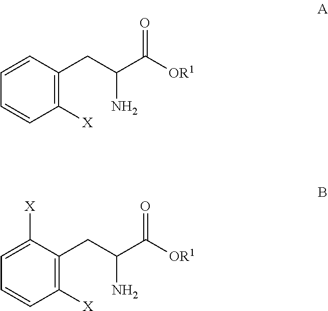
wherein R¹ is a hydrogen atom, a benzyl group or a C₁-C₆ linear or branched alkyl group, and X is a halogen atom selected from a group consisting of Cl, Br and I, by reaction of the compound of formula (I): 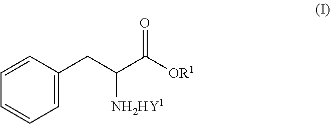
wherein R¹ is a hydrogen atom, and HY¹ is an acid,
with a halogenating agent, in the presence of a palladium catalyst and an acid HY², in an organic solvent or a mixture of organic solvents.

2. The process according to claim 1, wherein the compounds of formula (I), (A) and (B) are in (S) configuration.

3. The process according to claim 1, wherein R¹ is methyl or ethyl.

4. The process according to claim 1, wherein HY¹ is trifluoroacetic acid.

5. The process according to claim 1, wherein X is Cl or Br.

6. The process according to claim 5, wherein the halogenating agent is one selected from a group consisting of N-bromosuccinimide, N-chlorosuccinimide, N-bromophtalimide and N-chlorophtalimide.

7. The process according to claim 1, wherein the palladium catalyst is used in a sub stoichiometric amount.

8. The process according to claim 1, wherein the palladium catalyst is palladium(II) acetate.

9. The process according to claim 1, wherein the amount of the palladium catalyst is from 5 to 20 mol %.

10. The process according to claim 1, wherein HY² is trifluoroacetic acid.

11. The process according to claim 1, wherein the reaction is conducted in the presence of copper (II) acetate.

12. The process according to claim 1, wherein the temperature of the reaction is from 30° C. to 80° C.

13. The process according to claim 1, wherein the organic solvent is a mixture of dichloromethane and hexafluoroisopropanol.

14. The process according to claim 1, wherein the organic solvent is trifluoroacetic acid.

15. The process according to claim 1, wherein a mixture of the compounds (A) and (B) is obtained and subsequently separated into the compounds (A) and (B).

16. The process according to claim 1, further comprising a step of intramolecular aryl amination reaction, to lead to the compound of formula (IIIA), to the compound of formula (IIIB) or to a mixture thereof: 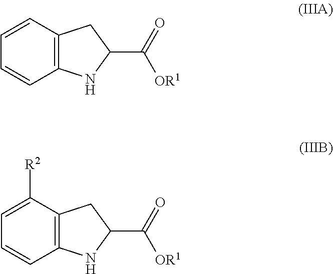
wherein R¹ is a hydrogen atom, a benzyl group or a C₁-C₆ linear or branched alkyl group, and R² is H, Cl, Br or I, followed, if the compound of formula (IIIB) is obtained, by a reduction reaction, to lead to the compound of formula (IIIA).
